(19) United States
(12) Patent Application Publication
(10) Pub. No.: US 2006/0149221 A1
(43) Pub. Date: Jul. 6, 2006

(54) Transaxillary breast augmentation instrument and method

(75) Inventors: Ghen Sugimoto, Miami, FL (US); Tania Choquehaunca, Miami, FL (US); Brad Herman, Miami, FL (US)

Correspondence Address: NIXON & VANDERHYE, PC, 901 NORTH GLEBE ROAD, 11TH FLOOR, ARLINGTON, VA, 22203, US

(73) Assignee: The University of Miami, Miami, FL (US)

(21) Appl. No.: 11/227,434

(22) Filed: Sep. 16, 2005

Related U.S. Application Data
(60) Provisional application No. 60/610,192, filed on Sep. 16, 2004.

(51) Int. Cl. A61B 17/00

(52) U.S. Cl. 606/1

(57) ABSTRACT

A tool and method for holding a breast implant during implantation in an endoscopic transaxillary breast augmentation procedure, to facilitate its proper alignment. The forceps style instrument holds the implant while accommodating the inflation tube to facilitate the proper placement of the implant, to minimize trauma to the patient and expedite the implantation and alignment process.

### CROSS-REFERENCE TO RELATED APPLICATION

This application claims the benefit of U.S. Provisional Application No. 60/610,192, which was filed on Sep. 16, 2004, the disclosure of which is incorporated herein by this reference.

### BACKGROUND OF THE INVENTION

The present invention relates to tools for facilitating breast augmentation surgery and more specifically to a transaxillary breast augmentation tool designed to ease the insertion of breast implants during the endoscopic transaxillary breast augmentation procedure.

The transaxillary breast augmentation procedure requires that a two to three inch incision be made under the armpit (transaxillary area). A cavity is then defined between the adjacent layers of the pectoral muscle using an endoscope and electro-surgical instruments. Once the cavity has been formed, a breast implant is inserted into the cavity, aligned, and filled, e.g., with saline. The implant frequently becomes twisted or folded during insertion. When this happens, it is necessary for the surgeon to remove the implant and reinsert it until it is correctly aligned.

### BRIEF DESCRIPTION OF THE INVENTION

A tool is provided in accordance with the invention to hold the implant during implantation and to facilitate its proper alignment. More specifically, the invention provides a forceps style instrument to hold the implant while accommodating the inflation tube to facilitate the proper placement of the implant, to minimize trauma to the patient and expedite the implantation and alignment process.

Thus, the invention may be embodied in a transaxillary breast augmentation tool to facilitate the insertion of a breast implant during an endoscopic transaxillary breast augmentation procedure, comprising: first and second arms each said arm including a distal portion and proximal portion, said distal and proximal portions of each said arm being defined in one of a common plane and generally parallel planes, and proximal ends of said proximal portions of said arms being hingedly coupled together, wherein a distal end of at least one of said arms is forked to define first and second tines and a rounded slot therebetween.

The invention may also be embodied in a transaxillary breast augmentation tool to facilitate the insertion of a breast implant during an endoscopic transaxillary breast augmentation procedure, comprising: first and second arms each said arm including a distal portion and proximal portion, said distal and proximal portions of each said arm being defined in one of a common plane and generally parallel planes, a longitudinal axis of said distal portion being disposed at an angle of greater than or equal to about 110 degrees and less than or equal to 160 with respect to a longitudinal axis of said proximal portion, and wherein proximal ends of said proximal portions of said arms being hingedly coupled together.

The invention may further be embodied in a method of inserting a prosthetic implant into a cavity formed in the body comprising: providing a tool including first and second arms each said arm including a distal portion and proximal portion, proximal ends of said proximal portions of said arms being hingedly coupled together, wherein a distal end of at least one of said arms is forked to define first and second tines and a rounded slot therebetween; disposing said implant between said distal portions of said arms, said implant including an inflation tube; disposing said inflation tube to extend through said rounded slot and generally proximally along said at least one arm; inserting said tool with said implant grasped between said arms through an incision to insert said implant into said cavity; spacing said distal portions of said arms to release said implant; withdrawing said instrument through said incision; and closing said incision.

The invention may also be embodied in a method of inserting a prosthetic implant into a cavity formed in the body comprising: providing a tool including first and second arms each said arm including a distal portion and proximal portion, proximal ends of said proximal portions of said arms being hingedly coupled together, said distal and proximal portions of each said arm being defined in one of a common plane and generally parallel planes, a longitudinal axis of said distal portion being disposed at an angle of greater than or equal to about 110 degrees and less than or equal to 160 with respect to a longitudinal axis of said proximal portion; disposing said implant between said distal portions of said arms; inserting said tool with said implant through an incision to insert said implant into said cavity; spacing said distal portions of said arms to release said implant and withdrawing said instrument through said incision; and closing said incision.

### BRIEF DESCRIPTION OF THE DRAWINGS

These and other objects and advantages of this invention, will be more completely understood and appreciated by careful study of the following more detailed description of the presently preferred exemplary embodiments of the invention taken in conjunction with the accompanying drawings, in which:

FIG. 1 is a plan view of a first side of a tool embodying the invention;

FIG. 2 is a plan view of the second side of the tool illustrated in FIG. 1;

FIG. 3 is an elevational view of an assembled tool embodying the invention in its closed and locked configuration;

FIG. 4 is a view similar to FIG. 3 but showing the tool in its unlocked and open configuration for receiving an uninflated breast implant;

FIG. 5 is an enlarged view showing an alternate tool arm junction in an embodiment of the invention;

FIG. 6 is an enlarged schematic view showing a further alternate tool arm junction according to the invention;

FIG. 7 is an enlarged schematic view of the locking sleeve of FIGS. 3 and 4;

FIG. 8 is an enlarged view taken along line 8-8 of FIG. 3;

FIG. 9 is a schematic elevational view of an alternate implantation tool embodying the invention;

FIG. 10 is a schematic illustration depicting a tool in accordance with the invention holding a breast implant in advance of implantation; and

FIG. 11 is a view similar to FIG. 8 showing the implant folded or rolled about the tool arms for insertion through a transaxillary incision.

### DETAILED DESCRIPTION OF THE INVENTION

The present invention provides a forceps style tool for holding a breast implant during implantation. The tool is comprised of first and second arms 10, 12 coupled at or adjacent a proximal end 14 and disposed in side by side, generally parallel relation for grasping a breast implant therebetween. Each of the arms of the tool is comprised of a distal segment or portion 16, 18 for engaging the implant and a proximal segment or portion 20, 22 which may be used as a handle to manipulate and advance the tool distal end to place and align the implant.

In the illustrated embodiment, the distal portion and the proximal portion of each arm is defined generally in a common plane, but those portions are oriented so that their respective longitudinal axes are inclined to define an elbow 24, 26. The elbow allows the surgeon to insert the implant from a natural angle at which the patient is laying while keeping the filling tube in the necessary upright position both inside and outside the body. The elbow angle is in a range of about 110-160 degrees. In the presently preferred and illustrated embodiment, the distal portion and proximal portion of the arm are disposed at an angle of about 130 degrees.

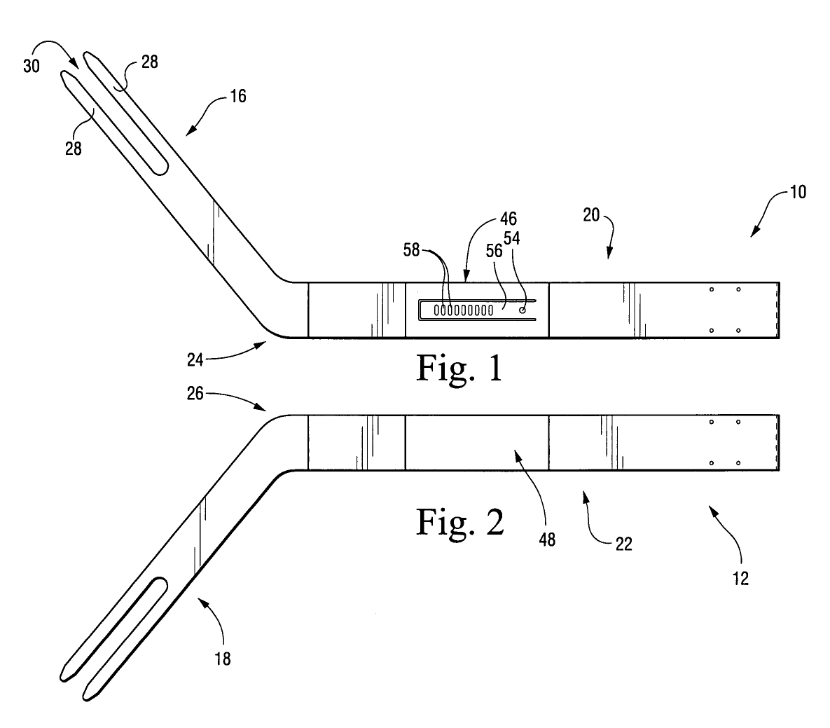

In an exemplary embodiment, the proximal and distal portions 20, 22 are generally rectangular. The proximal portions are about 8-10 inches, for example about 9 inches in length and about 1 inch wide while in a preferred embodiment, the distal portions 16, 18 are shorter and narrower, having a length of about 5-6 inches, for example 5.5 inches, and a width of about 0.8 inches. The length of the distal portion of the tool is important in this regard for inserting to the correct tissue depth. In the embodiment illustrated in FIGS. 1-4, the distal and proximal portions are integrated to define unitary arms. As discussed in greater detail below, however, the arms of the device may be formed from parts or segments that are secured together.

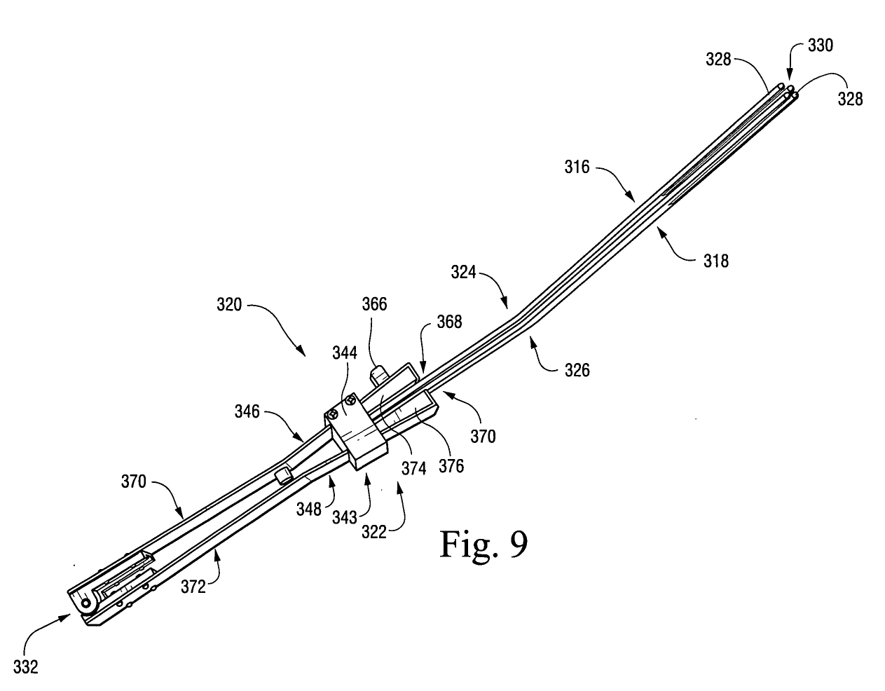

As illustrated in FIG. 9, on the ventral side of implant 50, a narrow tube 52 extends from a self-sealing valve. This is the filling tube used to flow saline into the implant. This tube frequently becomes twisted during the conventional implantation procedure. To address and solve this problem, in the presently preferred embodiment of the invention, the distal end of at least one of the arms of the tool is forked, comprising first and second tines 28, 328 with a tube slot 30, 330 defined therebetween to hold and align the filling tube 52 of the implant 50 during insertion. In an exemplary embodiment, the tines begin approximately 2.5 inches from the distal tip of the distal portion and thus are about half of the distal segment length. In an exemplary embodiment, the width of the tube slot is about 0.3 inches and the tine tips and groove are rounded to prevent any damage to the patent's tissue, to the implant, or to the inflation tube. As is apparent, the tool is used to grasp, insert and position, the implant while the angled configuration of the structure, as noted above, allows the surgeon to insert the implant from a natural angle while keeping the filling tube in the necessary upright position.

As mentioned above, the proximal ends of the arms of the tool are joined. In the embodiment illustrated in FIGS. 1-4, a hinge structure 32 is bolted to both of the flat bar pieces at the proximal end by, e.g., four threadless surgical grade stainless steel bolts 34. A spring structure 36 having first and second spring legs is disposed so that the legs extend from the hinge pin 42 along the portion of the length of each of the arms 10, 12. The spring hinge used is pre-adjusted to the appropriate springiness. Because the application of the tool does not require significant force and the little force that is necessary is applied by the surgeon and held by the locking mechanism, the hinge used in the embodiment illustrated in FIGS. 1-4 was designed so that the user cannot adjust the springiness or spring coefficient by tightening or loosening the bolts. In fact, the bolts are preferably riveted pins that are fixed in place.

As shown in FIG. 5, as an alternative to the hinge structure illustrated in FIGS. 1-4, the arms 110, 112 of the tool may be connected by forming the proximal portions of the arms as an integrated assembly so that the proximal end 114 of the tool is essentially a single piece of material folded in half to form proximal segments that are parallel to each other. In this embodiment, the resilience of the folded piece of material defines a spring structure 136 at the proximal end 114.

In yet a further embodiment, a hinge structure 232 is provided by first and second ears 238 formed integrally at the longitudinal side edges of one of the arms 210, adjacent the proximal end, for being disposed in side by side alignment with corresponding ears 240 of the other arm 212. A hinge pin 242 extends between the aligned ears to complete the hinge structure. A spring 236 may be disposed to encircle the hinge pin with spring legs extending along each of the arms of the tool, as illustrated in FIG. 6, so that the arms 210, 212 are resiliently urged apart.

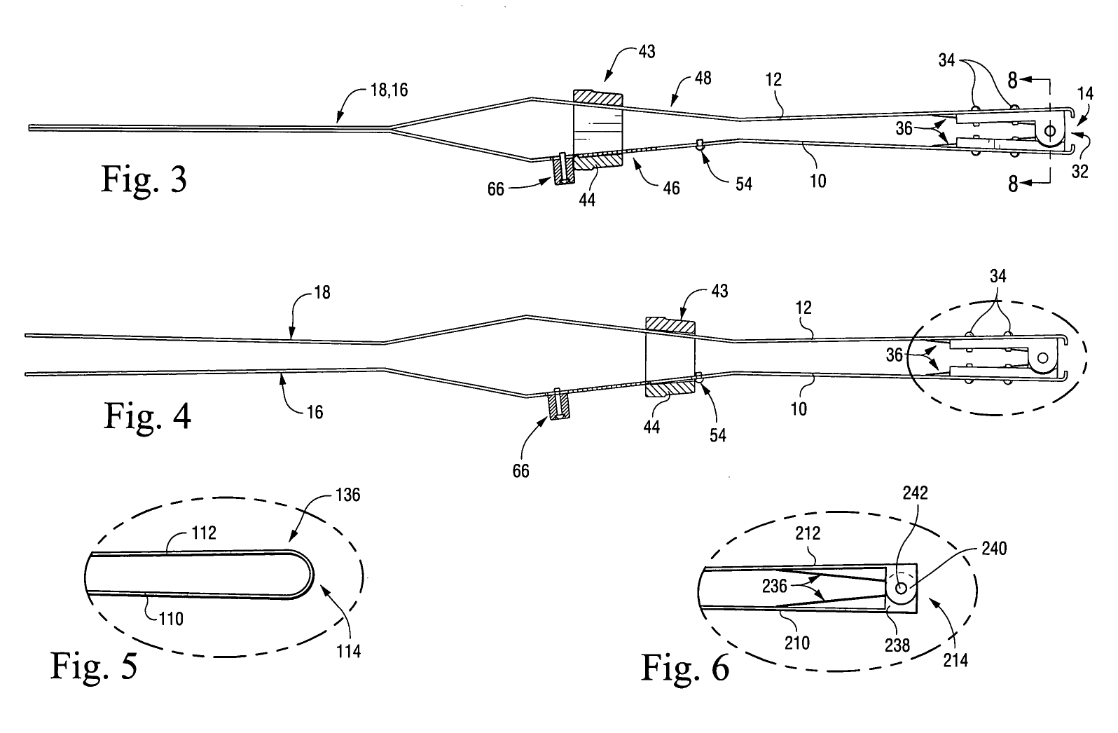

The embodiment described to have hinge 14 and 32 in FIGS. 3 and 4 or hinge 214 in FIG. 6 were developed to test varying spring coefficients and to allow for a changing tine design until the illustrated presently preferred tine length and configuration were discovered. While a mechanical hinge of the type illustrated in these figures may of course be used, it is presently contemplated that most advantageously, the embodiment of FIG. 5, wherein the two tines are connected as a solid piece would be preferred because the connected area will be pre-constructed with the appropriate spring coefficient.

In the presently proposed embodiment of the invention, a locking mechanism or assembly is provided to lock the arms of the instrument in their closed, implant grasping disposition, firmly grip the implant between the arms as the tool is manipulated to tunnel and place the implant in the formed cavity. In the illustrated embodiment, the locking mechanism 43 is provided on the proximal portion of the tool adjacent the elbow. An example of a suitable locking mechanism is a slide lock assembly including a locking sleeve 44 mounted in surrounding relation to the tool for engaging a cam surface defined along at least one of the arms for selectively camming the arm(s) into a closed configuration when the sleeve is moved distally. Conversely, the arms shift to an open configuration, responsive to the action of spring structure 36, 136, 236, when the sleeve is moved proximally.

Referring to the embodiment of FIGS. 3-4, the slide is disposed to slide from a proximal, unlocked position defined by a first stop (FIG. 4) to a more distal, locked position (FIG. 3). In this embodiment, the cam surface is defined by at least one arm being bent away from the other. Preferably, both arms are bent as at 46, 48 so that the arms 10, 12 of the tool are inclined away from each other. The motion of the locking sleeve along the inclined surfaces of the tool arms causes the distal portions 16-18 to clamp and hold the implant and hold it securely while it is tunneled to its deployment site. The locking sleeve fully encircles the tool arms 10, 12 to keep them in parallel side by side relation and thus prevent the arms from slipping laterally. In a preferred embodiment, a stop 54 is provided at the distal end of the locking assembly 43 to prevent undesired distal displacement of the locking sleeve.

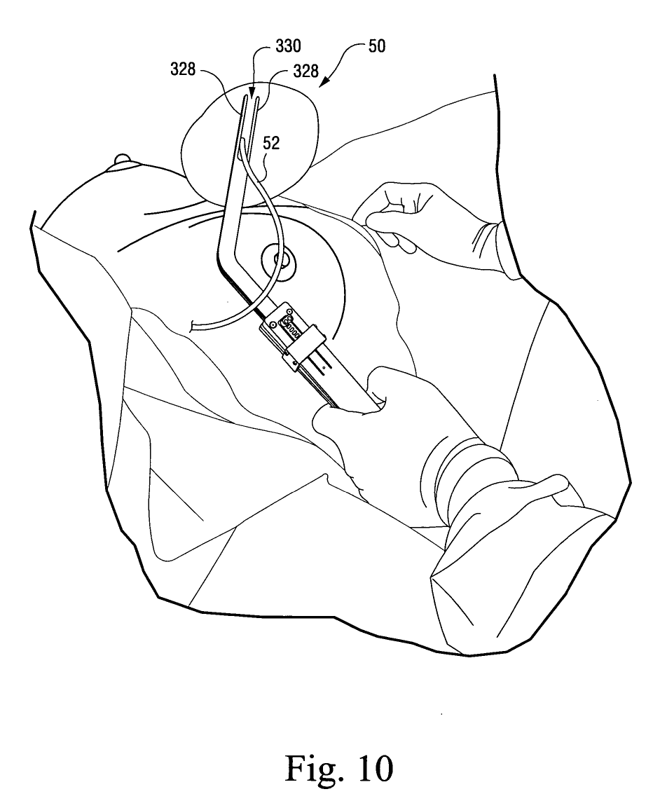

The preferred locked disposition of the distal portions 16, 18 of the tool may vary depending upon the implant 50 placed between the distal arm portions and whether it is held by the tool in its extended form, as illustrated, e.g., in FIG. 10, or after it has been rolled or folded. Thus, the locking assembly 43 preferably defines a plurality of locking positions.

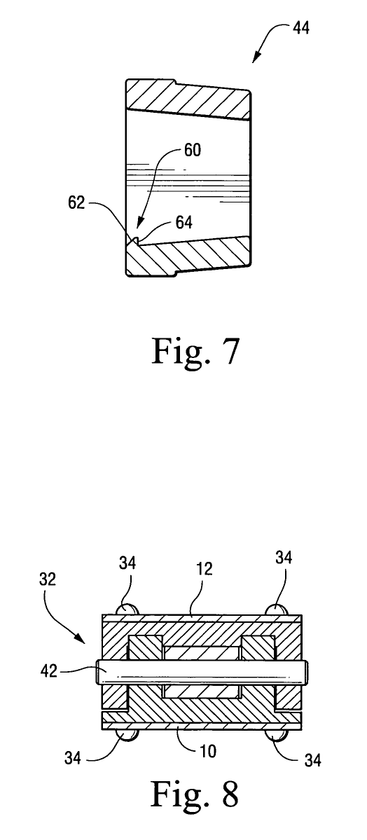

In an exemplary embodiment, the locking mechanism has nine levels or increments of locking tightness defining nine nominal spacings between the distal portions. In an exemplary embodiment, a resilient tongue 56 is formed in one of the tool arms and defines a plurality of grooves or cutouts 58 for receiving a complimentary locking projection 60 projecting from the locking sleeve. As illustrated in FIG. 7, the locking projection 60 has an inclined surface 62 and a perpendicular surface 64. Accordingly, as the locking sleeve is slid distally along the cam surfaces 46, 48, the distal portions 16, 18 of the tool arms 10, 12 move towards one another. Further, the locking projection 60 deflects the locking tongue 56 to in turn engage each of the locking receptacles 58. The inclined surface 62 facilitates deflection of the tongue, while the perpendicular surface 64 locks the locking projection 60 in a respective receptacle 58 to resist unintended proximal displacement. When the distal portions of the tool arms are thus brought into a desired proximate position, the locking sleeve projection is engaged with a respective groove or cutout and cannot slide proximally. A distal stop 66 may be provided to limit distal displacement of the locking sleeve 44. When it is desired to slide the locking sleeve proximally, to unlock the arms of the tool and release the implant 50, the locking tongue 56 may be manually depressed to release the locking projection 60 from the corresponding groove or cutout 58 and the locking sleeve 44 may be displaced proximally.

An alternate embodiment of the invention is illustrated in FIG. 9. In this embodiment, each arm of the tool is formed in two parts, the first part comprising the distal portion 316, 318; elbow 324, 326; and the distal end 368, 370 of the proximal portion 320, 322 of the tool. The remainder 372, 374 of the proximal portion 320, 322 of the tool is separately formed and includes therein the spring hinge assembly 332 and locking mechanism 343. The spring hinge assembly 332 of this embodiment corresponds to that illustrated and described above with reference to FIGS. 3 and 4. The locking mechanism 343 generally corresponds to the locking assembly 43 described above with reference to FIGS. 3 and 4, including a locking sleeve 44, inclined, camming surfaces 346, 348 and a distal stop 366 to limit displacement of the locking sleeve. In this embodiment, a filler support structure 374, 376 is disposed within and supports the inclined camming surfaces and defines a structure to which the more distal portions 368, 370 of the tool may be secured.

It is to be understood that while an exemplary locking mechanism has been illustrated and described, other known mechanisms for holding the arms in a selected implant clamping disposition may be provided to secure the arms in an implant grasping disposition.

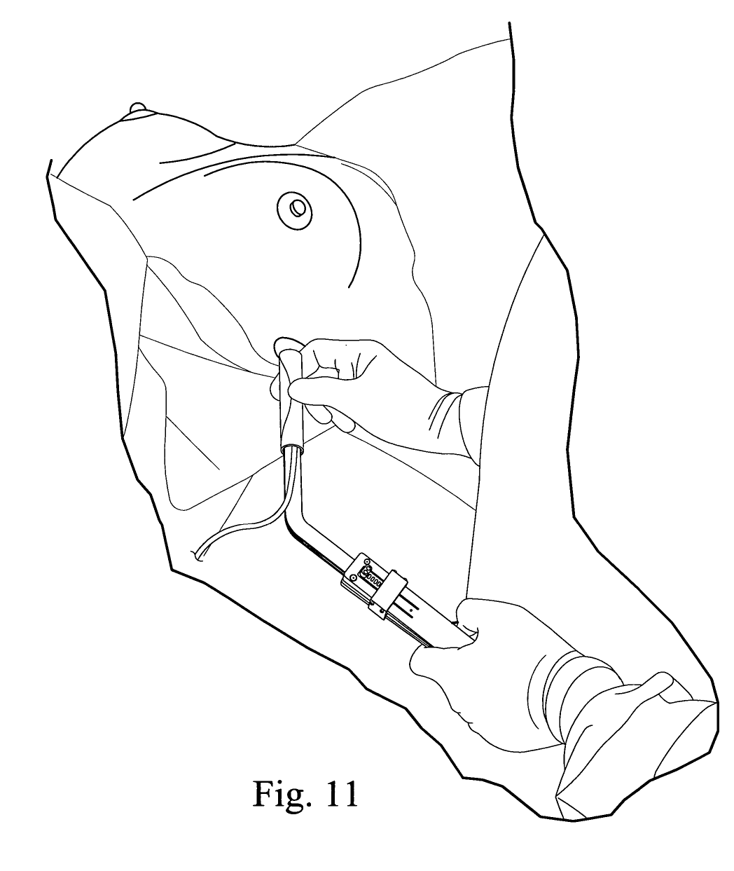

Referring to FIG. 11, the implant 50 can be rolled folded or just placed between the arms of the tool before the locking mechanism is applied. By way of non-limiting example, the implant is illustrated with a tool according to the FIG. 10 embodiment. When the implant is just placed between the arms of the instrument, the implant may be rolled or folded about the distal portions of the arms, as illustrated in FIG. 12. The grip on the implant is such that the implant will not be torn or ripped but is firm enough to guide it through the 2 inch incision in the transxillary area.

The transaxillary breast augmentation tool as described hereinabove is able to hold an implant in a steady position, with the filling tube lined up in the tube slot of the forked region, while the surgeon is aligning and inserting the implant into the prepared cavity. Due to the dual fork structure of the presently preferred embodiment (both arms of the instrument being forked) the tool is capable of inserting implants under both the right and left breast.

In a presently preferred embodiment, the tool is formed from stainless steel so that it can be sterilized and reused. It is also possible, however, for the tool to be formed from thermo-injected plastic that can be sterilized and thus have the added advantages of inexpensive manufacture, light weight for manipulation, and disposability. Such a disposable “one-time use” tool could be pre-packaged with the implant.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method of inserting a prosthetic implant into a cavity formed in the body comprising:
providing a tool including first and second arms each said arm including a distal portion and proximal portion, proximal ends of said proximal portions of said arms being hingedly coupled together, wherein a distal end of at least one of said arms is forked to define first and second tines and a rounded slot therebetween;
disposing said implant between said distal portions of said arms, said implant including an inflation tube;
disposing said inflation tube to extend through said rounded slot and generally proximally along said at least one arm;
inserting said tool with said implant grasped between said arms through an incision to insert said implant into said cavity;
spacing said distal portions of said arms to release said implant;
withdrawing said instrument through said incision; and
closing said incision

2. A method as in claim 1, further comprising flowing biocompatible fluid through said tube to inflate said implant before said closing step.

3. A method as in claim 1, further comprising disengaging and withdrawing said inflation tube before said closing step.

4. A method as in claim 1, wherein said distal and proximal portions of each said arm are defined in one of a common plane and generally parallel planes, a longitudinal axis of said distal portion being disposed at an angle of greater than or equal to about 110 degrees and less than or equal to 160 with respect to a longitudinal axis of said proximal portion.

5. A method as in claim 4, wherein said angle is 130 degrees.

6. A method as in claim 1, wherein said tool further comprises a locking mechanism for selectively locking said distal portions proximate one another to grasp an implant therebetween and further comprising, before said inserting, locking said distal portions in position to grasp said implant therebetween.

7. A method as in claim 1, wherein said implant is a breast implant.

8. A method as in claim 7, wherein said incision is a transaxillary incision.

9. A method of inserting a prosthetic implant into a cavity formed in the body comprising:
providing a tool including first and second arms each said arm including a distal portion and proximal portion, proximal ends of said proximal portions of said arms being hingedly coupled together, said distal and proximal portions of each said arm being defined in one of a common plane and generally parallel planes, a longitudinal axis of said distal portion being disposed at an angle of greater than or equal to about 110 degrees and less than or equal to 160 with respect to a longitudinal axis of said proximal portion;
disposing said implant between said distal portions of said arms;
inserting said tool with said implant through an incision to insert said implant into said cavity;
spacing said distal portions of said arms to release said implant and withdrawing said instrument through said incision; and
closing said incision.

10. A method as in claim 9, wherein said angle is 130 degrees.

11. A method as in claim 9, wherein said tool further comprises a locking mechanism for selectively locking said distal portions proximate one another to grasp an implant therebetween and further comprising, before said inserting, locking said distal portions in position to grasp said implant therebetween.

12. A method as in claim 9, wherein said implant is a breast implant.

13. A method as in claim 12, wherein said incision is a transaxillary incision.

14. A transaxillary breast augmentation tool to facilitate the insertion of a breast implant during an endoscopic transaxillary breast augmentation procedure, comprising:
first and second arms each said arm including a distal portion and proximal portion, said distal and proximal portions of each said arm being defined in one of a common plane and generally parallel planes, and proximal ends of said proximal portions of said arms being hingedly coupled together, wherein a distal end of at least one of said arms is forked to define first and second tines and a rounded slot therebetween.

15. A tool as in claim 14, wherein said hinged coupling comprises a spring for urging said distal portions of said arms into a spaced apart relation.

16. A tool as in claim 14, wherein said tool further comprises a locking mechanism for selectively locking said distal portions proximate one another to grasp an implant therebetween.

17. A tool as in claim 16, wherein said locking mechanism comprises a locking sleeve mounted in surrounding relation to the arms and displaceable along a portion of the length thereof, said locking sleeve engaging an inclined surface defined along a portion of at least one of the arms to selectively urge the distal portions of the arms toward one another when the sleeve is moved distally.

18. A tool as in claim 14, wherein said hinged coupling comprises first and second ears defined at a proximal end of each of said arms for being disposed in side by side facing relation to each other and a hinge pin therebetween.

19. A tool as in claim 14, wherein said proximal portions of said arms are integrally formed to define a U-shaped at said proximal end.

20. A transaxillary breast augmentation tool to facilitate the insertion of a breast implant during an endoscopic transaxillary breast augmentation procedure, comprising:
first and second arms each said arm including a distal portion and proximal portion, said distal and proximal portions of each said arm being defined in one of a common plane and generally parallel planes, a longitudinal axis of said distal portion being disposed at an angle of greater than or equal to about 110 degrees and less than or equal to 160 with respect to a longitudinal axis of said proximal portion, and wherein proximal ends of said proximal portions of said arms being hingedly coupled together.

21. A tool as in claim 20, wherein said hinged coupling comprises a spring for urging said distal portions of said arms into a spaced apart relation.

22. A tool as in claim 20, wherein said tool further comprises a locking mechanism for selectively locking said distal portions proximate one another to grasp an implant therebetween.

23. A tool as in claim 22, wherein said locking mechanism comprises a locking sleeve mounted in surrounding relation to the arms and displaceable along a portion of the length thereof, said locking sleeve engaging an inclined surface defined along a portion of at least one of the arms to selectively urge the distal portions of the arms toward one another when the sleeve is moved distally.

24. A tool as in claim 20, wherein said hinged coupling comprises first and second ears defined at a proximal end of each of said arms for being disposed in side by side facing relation to each other and a hinge pin therebetween.

25. A tool as in claim 20, wherein said proximal portions of said arms are integrally formed to define a U-shaped at said proximal end.
